(19) United States
(12) Patent Application Publication
(10) Pub. No.: US 2024/0325790 A1
(43) Pub. Date: Oct. 3, 2024

(54) SOLID PHANTOM DEVICE FOR BEAM SCANNING

(71) Applicant: The University of Toledo, Toledo, OH (US)

(72) Inventors: E. Ishmael Parsai, Toledo, OH (US); Diana Shvydka, Toledo, OH (US); Kanru Xie, Toledo, OH (US)

(73) Assignee: The University of Toledo, Toledo, OH (US)

(21) Appl. No.: 18/580,987

(22) PCT Filed: Jul. 21, 2022
(86) PCT No.: PCT/US2022/037793

§ 371 Date: Jan. 19, 2024

Related U.S. Application Data
(60) Provisional application No. 63/224,685, filed on Jul. 22, 2021.
(60) Provisional application No. 63/224,608, filed on Jul. 22, 2021.

(51) Int. Cl. A61N 5/10

(52) U.S. Cl. CPC A61N 5/1071; A61N 2005/1076

(57) ABSTRACT

Provided is a device comprising a phantom comprising a solid water material, having a square cross-sectional shape, and having a width that varies monotonically along a height of the solid phantom; and an array of radiation detectors disposed within the phantom; wherein the array of radiation detectors is configured to detect radiation within the phantom. Further provided is a linear accelerator having a gantry and comprising the device as described herein installed in a treatment head of the gantry. In certain embodiments, the linear accelerator further comprises software to interface the device with the linear accelerator.

### RELATED APPLICATIONS

This application claims priority to U.S. Provisional Application No. 63/224,608 filed under 35 U.S.C. § 111 (b) on Jul. 22, 2021, as well as U.S. Provisional Application No. 63/224,685 filed under 35 U.S.C. § 111 (b) on Jul. 22, 2021, the disclosures of which are incorporated herein by reference in their entireties for all purposes.

### STATEMENT REGARDING FEDERALLY SPONSORED RESEARCH

This invention was made with no government support. The government has no rights in this invention.

### BACKGROUND

Radiation therapy is a common curative procedure to treat cancer. The goal of radiation therapy is to expose the tumor to a sufficient dose of radiation so as to eradicate all cancer cells. The radiation dose is often close to the tolerance level of the normal body tissues. Therefore, it is necessary to determine the dosage levels in different parts of the irradiated body with high accuracy.

Characterization of a radiation beam is a major part of acceptance testing and commissioning of complex linear accelerator units which are used for radiation treatment of cancer patients. Beam scanning using a computerized water phantom is a common practice to conduct acceptance testing and commissioning of new x-ray producing linear accelerators, as well as for periodic quality assurance tests including the annual calibrations, and after any repair that may have affected the beam parameters. A typical computerized 3D water scanning system involves a very delicate piece of equipment that works with ionization chambers, and comes with a few auxiliary parts, including at least two small volume ionization chambers, triax cables (special cables for measurement of charge), a large acrylic tank that will contain water and can be aligned under the radiation beam, and often a jack system on the wheel to allow adjusting the height and position of the tank and water surface relative to the source of radiation. In addition to the hardware, a specialized software package comes with the system to automatically drive the ionization chambers inside the water tank from outside the treatment room to measure the beam characteristics under different configurations. This whole assembly is typically in the order of half a million dollars or more and as a delicate system needs to be handled with extreme care. In some cases, the tank dimensions are 675× 645×560 mm, with a scanning volume of 480×480×410 mm. In addition to having a learning curve for setup and proper operation of this system, the required time spent for setup is approximately an hour if everything goes well and the operator has fluency in the system operation, and nearly 45 minutes is required for teardown. Moreover, this system cannot be too far from the linear accelerator room as delivering the unit back and forth could damage the system, which effects the accuracy and resolution in the acquired data. Therefore, it also requires space to keep it in the hospital near the treatment rooms.

There are also planar detector arrays that are used for beam scanning, though only to measure beam profiles at a fixed depth. However, no system other than computerized water scanning systems are available to answer all the clinical needs. Thus, there is a need in the art for new and improved systems for the accurate measurement of beam profiles and depth-dose curves.

### SUMMARY

Provided is a device comprising a phantom comprising a solid water material, having a square cross-sectional shape, and having a width that varies monotonically along a height of the solid phantom; and an array of radiation detectors disposed within the phantom; wherein the array of radiation detectors is configured to detect radiation within the phantom.

In certain embodiments, the width increases monotonically with the height in a direction of from a beam side surface to an opposing surface.

In certain embodiments, the phantom consists essentially of the solid water material.

In certain embodiments, the radiation detectors are diode detectors, metal-oxide-semiconductor field-effect transistors (MOSFETs), thermoluminescent dosimeter (TLD) chips, radiochromic films, or combinations thereof. In certain embodiments, the array of radiation detectors comprises diode detectors. In certain embodiments, each of the radiation detectors is a diode detector.

In certain embodiments, the array of radiation detectors comprises a radiation detector about every 1 cm in each plane of the phantom.

In certain embodiments, the array of radiation detectors comprises a radiation detector about every 1 cm in each plane of the phantom; and the array of radiation detectors comprises diode detectors.

In certain embodiments, the array of radiation detectors comprises a radiation detector about every 1 cm in each plane of the phantom; the array of radiation detectors comprises diode detectors; and the phantom consists essentially of the solid water material.

In certain embodiments, the array of radiation detectors comprises diode detectors; and the phantom consists essentially of the solid water material.

In certain embodiments, the phantom consists essentially of the solid water material; and the array of radiation detectors comprises a radiation detector about every 1 cm in each plane of the phantom.

In certain embodiments, the array of radiation detectors comprises a radiation detector about every 1 cm in each plane of the phantom; the array of radiation detectors comprises diode detectors; and the phantom consists essentially of the solid water material.

In certain embodiments, the solid water material comprises 2.9-3.3% w/w glass micro bubbles, 60-90% w/w epoxy, acrylic, or polyurethane, 3-5% w/w CaCO₃, 1-3% w/w MgO, and 8-12% w/w polyethylene. In certain embodiments, the solid water material comprises 3.09% w/w glass micro bubbles, 57.88% w/w araldite, 23.15% w/w jeffamine, 3.89% w/w CaCO₃, 1.80% w/w MgO, 9.98% w/w polyethylene, and 0.2% w/w Na₅Al₆Si₆O₂₄S₄ or Si₄O₁₀ (OH)₂Mg₃—Co₃Ca—Al, with an elemental composition of 65.81% w/w carbon, 19.36% w/w oxygen, 8.14% w/w hydrogen, 2.21% w/w nitrogen, 1.78% w/w calcium, 1.14% w/w silicon, and 1.11% w/w magnesium.

In certain embodiments, the device is configured to be inserted within a head of a gantry of a linear accelerator.

Further provided is a linear accelerator having a gantry and comprising the device as described herein installed in a treatment head of the gantry. In certain embodiments, the linear accelerator further comprises software to interface the device with the linear accelerator.

Further provided is a method for analyzing a dose response depth or a profile of a beam from a linear accelerator, the method comprising irradiating the beam from a linear accelerator into the device of claim 1 and detecting the beam with the array of radiation detectors to obtain dose response depth or profile data from the beam. In certain embodiments, the method further comprises comparing the obtained dose response depth or profile data to a treatment plan for a patient. In certain embodiments, the array of radiation detectors comprises a radiation detector about every 1 cm in each plane of the phantom; the array of radiation detectors comprises diode detectors; and the phantom consists essentially of the solid water material.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1: Perspective view of a non-limiting example embodiment of a solid water phantom device in accordance with the present disclosure.

FIG. 2: Illustration of a linear accelerator including a solid water phantom device in the gantry head.

FIG. 3: Monte Carlo N-Particle Code (MCNP) computed setup of Varian Edge LINAC and the PDD phantom attached to the gantry head at 60 cm from the source.

FIG. 4: MCNP setup of Varian Edge LINAC and the profiles phantom attached to the gantry head at 60 cm from the source.

FIG. 5: Percent depth dose computed with the virtual LINAC and phantom compared to Wellhoffer water scanning phantom.

FIG. 6: Computed X-plane profile at 10 cm depth using a 6FFF X-ray beam and the virtual LINAC compared to measured data in Wellhoffer water scanning phantom.

FIG. 7: Computed Y-plane profile at 10 cm depth using a 6FFF X-ray beam and the virtual LINAC compared to measured data from the Wellhoffer water scanning phantom.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Provided herein is a water-mimicking solid phantom device for beam scanning to replace computerized 3D water scanning systems. The device is a phantom system manufactured from solid water that contains arrays of detectors in a special geometrical shape and that allows measurement of beam profiles at any desired depth or direction for all the photon and electron beams. The device also allows measurement of percent depth dose, PDD, for all the clinical energies. The entire assembly may be attached to the head of the gantry of a linear accelerator (LINAC), and may completely eliminate the need for computerized water scanning systems.

In accordance with the present disclosure, provided is a solid phantom that is capable of replacing the computerized water scanning system in its entirety, and is easy to install at the head of the gantry. Furthermore, within a few minutes of data collection, the user can extract profiles and percent depth dose data through software associated with the solid phantom. This eliminates hours of water scanning setup and teardown, and a long wait for accurate data collection through the slow movement of field ionization chamber inside the water tank, and eliminates errors caused by potential wrong setups or driving of the scanning system. As shown in the examples herein, the solid phantom shows excellent agreement compared to measured data using a water phantom.

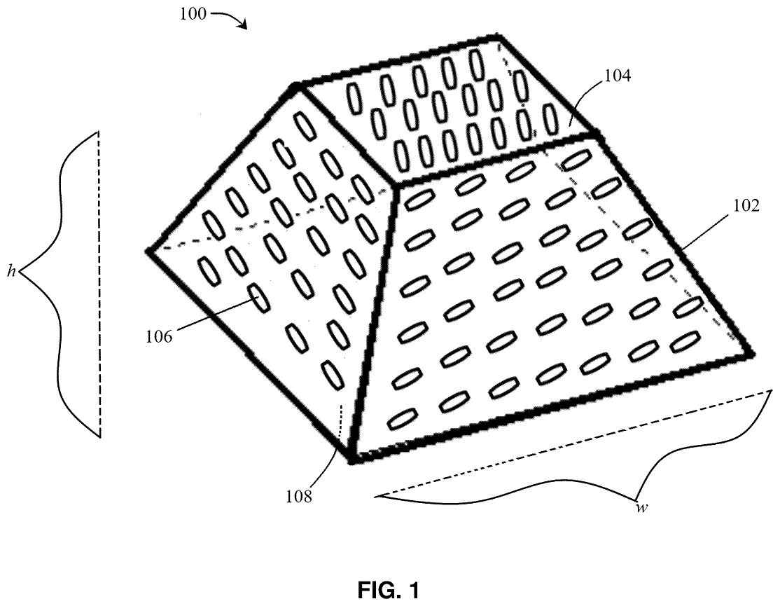

Referring now to FIG. 1, an example device 100 is depicted. The device 100 includes a phantom 102 having a square cross section on a beam side surface 104. The phantom 102 has an opposing surface 108 that may also have a square cross section. The phantom 102 may have a width w that changes with the height h of the phantom 102. The device 100 may be in the shape of a frustum, such as a frustum of a square pyramid. In some embodiments, the width w changes monotonically with the height h. In some examples, the width w monotonically increases with the height h in the direction of from the beam side surface 104 to the opposing surface 108. Advantageously, the square cross-sectional shape combined with the width w that changes in a monotonic manner as a function of the height h of the phantom 102 allows for the phantom 102 to accommodate the divergence of the beam from a linear accelerator. Other shapes of the phantom 102 are possible and encompassed within the scope of the present disclosure. However, other shapes may be less ideal. For example, a cone shape is problematic because the head of the gantry gives off a square-shaped beam, and so a cone shape may not capture the entire beam.

Referring still to FIG. 1, the phantom 102 may have any suitable dimensions based on the size and configuration of the linear accelerator the phantom 102 is to be used with. In one non-limiting example, the phantom 102 has a height h of about 40 cm, a width w at the beam side surface 104 of about 6 cm, and a width w at the opposing surface 108 of about 10 cm. In another non-limiting example, the phantom 102 has a height h of about 16.8 cm, a width w at the beam side surface 104 of about 24 cm, and a width w at the opposing surface 108 of about 22.4 cm. However, many other sizes are possible and encompassed within the scope of the present disclosure.

Referring still to FIG. 1, the phantom 102 is generally solid, and may be composed of a solid water material. The solid water material is a solid material that mimics water by having a similar electron density to that of water. The solid water material may be, for example, a composition as described in U.S. Pat. No. 9,669,116, which is incorporated herein by reference. Solid water material may include, for example, glass micro bubbles, araldite, jeffamine, magnesium oxide, and polyethylene, or in another example, may include glass micro bubbles, an epoxy, acrylic, or polyretheane, and polyethylene. The solid water material may result in a composition having an elemental composition that includes carbon, oxygen, hydrogen, nitrogen, calcium, and magnesium. The solid water material may further include one or more pigments. In one non-limiting example, the solid water material is composed of 2.9-3.3% w/w glass micro bubbles, 60-90% w/w epoxy, acrylic, or polyurethane, 3-5% w/w CaCO₃, 1-3% w/w MgO, and 8-12% w/w polyethylene. In another non-limiting example, the solid water material is composed of 3.09% w/w glass micro bubbles, 57.88% w/w araldite, 23.15% w/w jeffamine, 3.89% w/w CaCO₃, 1.80% w/w MgO, 9.98% w/w polyethylene, and 0.2% w/w Na₅Al₆Si₆O₂₄S₄ or Si₄O₁₀ (OH)₂Mg₃—Co₃Ca—Al, with an elemental composition of 65.81% w/w carbon, 19.36% w/w oxygen, 8.14% w/w hydrogen, 2.21% w/w nitrogen, 1.78% w/w calcium, 1.14% w/w silicon, and 1.11% w/w magnesium. However, many other solid water materials are possible and encompassed within the scope of the present disclosure.

Referring still to FIG. 1, the device 100 may include an array of radiation detectors 106. In some embodiments, the radiation detectors 106 are diode detectors. Diode detectors are very reliable detectors that have a high resistance to radiation damage. The radiation detectors 106 can be arranged to accommodate all the available energies in a linear accelerator including all the photon and electron beams. The array of radiation detectors 106 may include a radiation detector 106 about every 1 cm in each plane of the phantom 102. However, while this spacing of radiation detectors 106 results in good resolution, other spacing of the radiation detectors 106 is possible and encompassed within the scope of the present disclosure. Furthermore, though diode detectors are described as example radiation detectors 106, any type of radiation detector can be utilized, including, but not limited to, metal-oxide-semiconductor field-effect transistors (MOSFETs), thermoluminescent dosimeter (TLD) chips, or radiochromatic films. Moreover, in some embodiments, the array of radiation detectors 106 may include multiple kinds of radiation detectors 106, such that, for example, some of the radiation detectors 106 are diode detectors and some of the radiation detectors 106 are MOSFETs, TLD chips, or radiochromatic films. In some embodiments, the radiation detectors 106 collect data in three dimensions simultaneously.

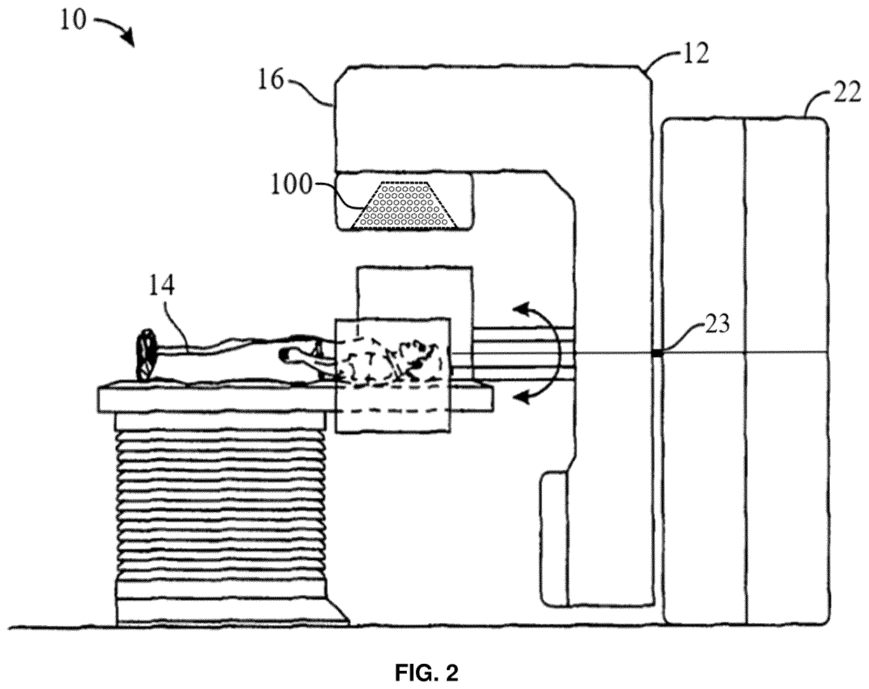

A connection between the treatment machine and the radiation detectors 106 and/or a connection to a control PC, such as through a local area network, may be established. The radiation detectors 106 may communicate wirelessly with a computer or LINAC system, or otherwise may be hardwired together and to a computer or the LINAC system. For ease of illustration, wiring of the radiation detectors 106 is omitted in FIGS. 1-2.

Referring now to FIG. 2, the device 100 may be installed within the head of the gantry 12 of a linear accelerator 10. An example linear accelerator 10 may include a rotatable gantry 12 to place a patient 14 and the target volume within the range of operation for the radiation treatment head 16. The rotatable gantry 12 may be pivotably attached to a drive stand 22 through a suitable joint 23. The gantry 12 may take the form of a C-shaped arm, and may include internal components such as an electron gun and an accelerator structure. The radiation treatment head 16 may be a radiation source generally in the 4 MV to 25 MV energy range, for example, at 6 MV. The collision of electrons with a high density transmission target within the treatment head 16 creates the X-rays (photons), forming a forward peaking shaped X-ray beam in the direction of the patient's tumor. The head 16 of the gantry 12 typically includes a space configured to hold a block or other apparatus, and the device 100 may be conveniently inserted into such a space. By inserting the device 100 into the head 16 of the gantry 12, the device 100 is positioned in the path of the beam. Suitable electronics for interfacing the device 100 and the linear accelerator 10 may be similarly installed in the gantry 12. Though FIG. 2 depicts a patient 14, it is understood that the device 100 may be removed from the gantry 12 prior to performing a treatment on the patient 14.

Advantageously, the device 100 is small enough to be lifted up by one person. The device 100 is easily installed at the head of the gantry 12, and within a few minutes of data collection, the user can extract profiles and percent depth dose (PDD) data quickly through associated software. This significantly reduces the amount of time necessary for setup and PDD data collection compared to using a conventional water system. The device 100 can save time in data acquisition, and provide an accurate dosimetry evaluation while providing convenience and efficiency in equipment setup.

Pre-treatment verification can be performed using the device 100. The device 100 can produce information regarding a dose distribution within the phantom 102 by measuring the beam with the array of radiation detectors 106. The measurement can be divided into time intervals, and the information can be used in treatment validation. The measurements made by the radiation detectors 106 are read by a physicist, and can be compared to a treatment plan.

### EXAMPLES

Using Monte Carlo simulations, a LINAC (Varian Edge 6FFF) was modeled in MCNP5, and a PDD (10×10 cm²) was simulated and confirmed to give profiles at different depths, which agreed favorably with measured data acquired in a 3D water tank (40×40×40 cm³). A virtual solid device placed at 60 cm source to surface distance (SSD) was then modeled on the LINAC, which is near the closest distance to the gantry head. The device modeled in MCNP was constituted of two phantoms that can be interlocked and are made of solid water material with much smaller volume and geometrical shape to accommodate acquisition of data for PDD and profiles. Scoring points were placed in the virtual phantom representing arrays of diode detectors for the measurement of PDD and profiles. Specifically, the phantom had in every 5 mm distance in X, Y, and Z directions a scoring point to mimic a diode detector placement for measuring the dose. Data were acquired through simulation and compared with data from the 3D water tank.

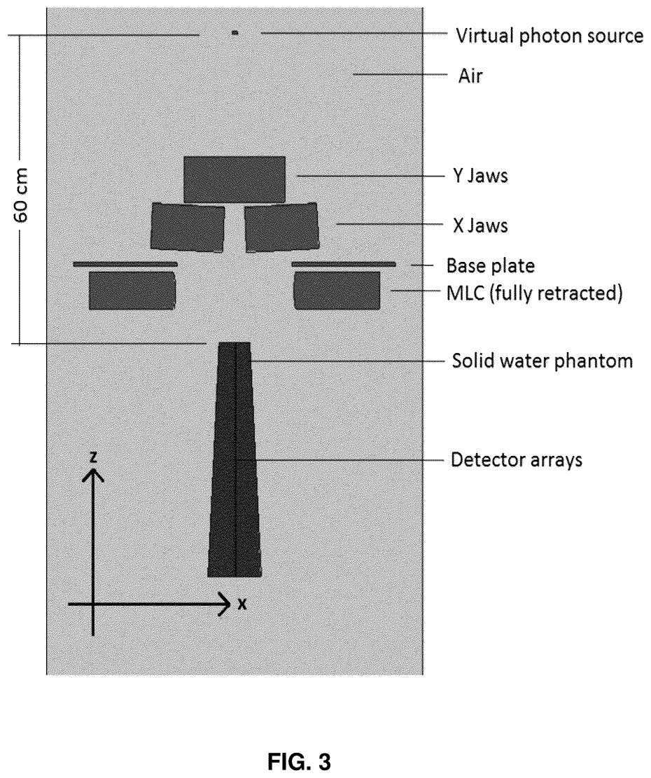

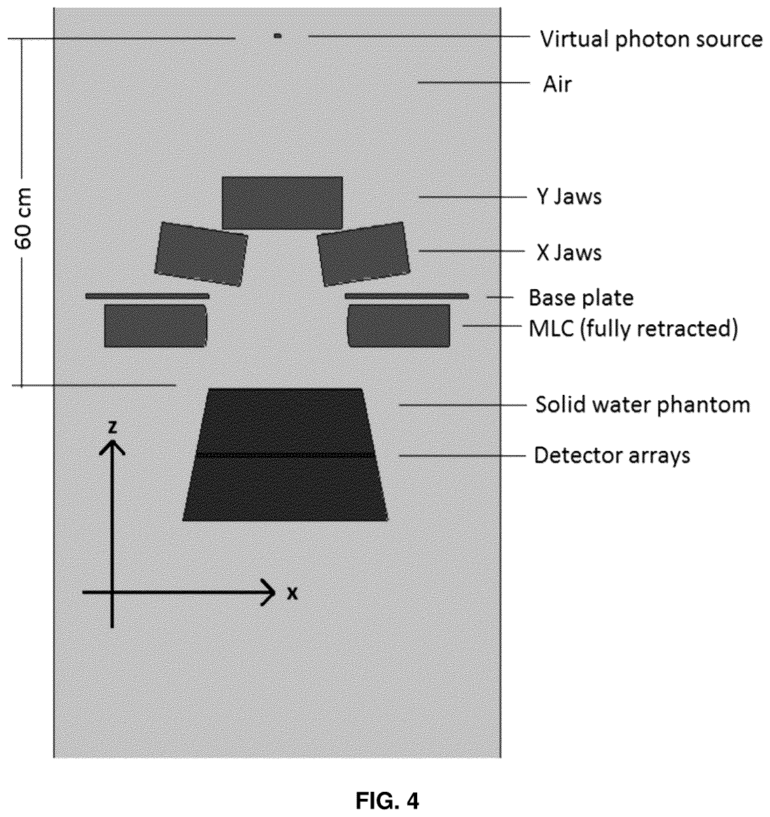

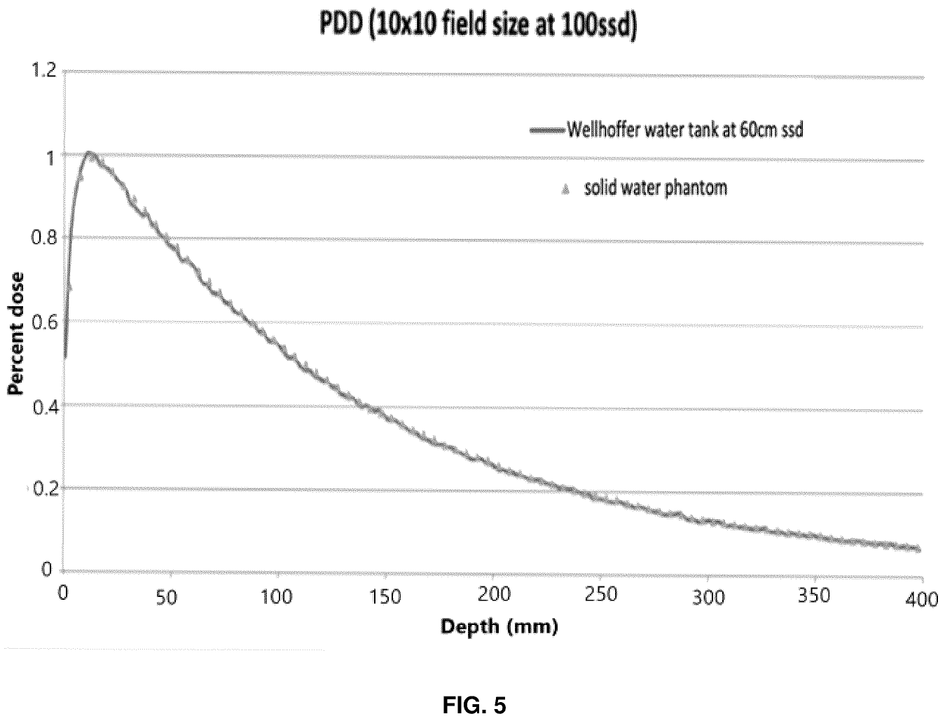

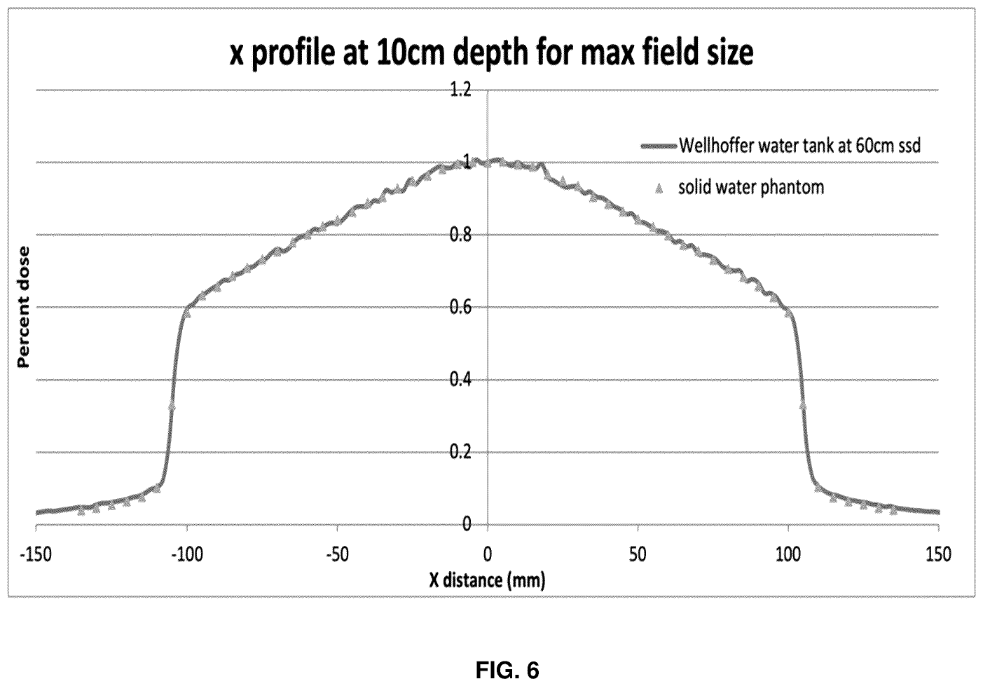

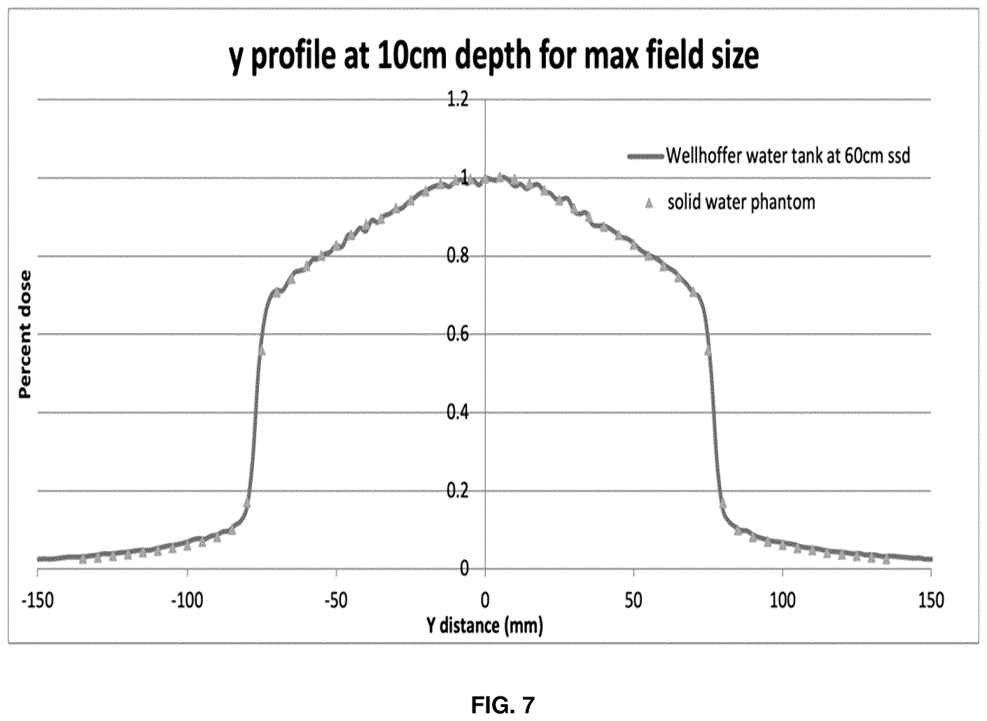

The virtual LINAC model was previously validated by comparing to machine commissioning measured data acquired from a standard Wellhoffer water scanning system. For this example, the tank was moved to 60 cm SSD in simulation, which is a distance not available for actual measurement due to the water tank dimension. FIG. 3 shows the MCNP setup of the virtual LINAC model of equivalent 10×10 cm² field size at 100 SSD for PDD simulation. The phantom was a slender frustum of a square pyramid with an array of diode detectors along the CAX. FIG. 4 shows the phantom for profiles measurement at max field size (22×30 cm²) for Varian Edge. The profiles phantom was a thin frustum of a square pyramid with an array of diode detectors at 10 cm depth. FIG. 5 shows the simulation results of PDD. FIG. 6 and FIG. 7 show crossline and inline profiles, respectively, comparing the data from the Wellhoffer water tank at 60 cm SSD. FIGS. 5-7 show excellent agreement compared to the measured data using a water phantom.

The simulated PDD and profiles in the solid water phantom agreed favorably with the data from the 3D water tank, within 1% error. The effective mass of the phantom for profiles is about 17 lbs., while the phantom for PDD is about 6 lbs., only a few percent of the mass of a 3D water tank, indicating much easier handling and setup for a solid water phantom as simulated compared to a conventional water tank.

This example shows the feasibility of a solid water phantom by comparing simulation data from the two solid water phantoms to data obtained from a conventional water tank, namely a Wellhoffer water phantom. Given the significantly reduced mass of the solid water phantom compared to a conventional water phantom system, this example shows that solid water phantom provides efficiencies in setup and use not realized with conventional water phantom systems.

Certain embodiments of the devices and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the devices and methods described herein to various usages and conditions. Various changes may be made, and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A device comprising:
a phantom comprising a solid water material, having a square cross-sectional shape, and having a width that varies monotonically along a height of the phantom; and
an array of radiation detectors disposed within the phantom;
wherein the array of radiation detectors is configured to detect radiation within the phantom.

2. The device of claim 1, wherein the array of radiation detectors comprises a radiation detector about every 1 cm in each plane of the phantom.

3. The device of claim 1, wherein the phantom consists essentially of the solid water material.

4. The device of claim 1, wherein the radiation detectors are diode detectors, metal-oxide-semiconductor field-effect transistors (MOSFETs), thermoluminescent dosimeter (TLD) chips, radiochromic films, or combinations thereof.

5. The device of claim 1, wherein the array of radiation detectors comprises diode detectors.

6. The device of claim 1, wherein each of the radiation detectors is a diode detector.

7. The device of claim 1, wherein:
the array of radiation detectors comprises a radiation detector about every 1 cm in each plane of the phantom; and
the array of radiation detectors comprises diode detectors.

8. The device of claim 1, wherein:
the array of radiation detectors comprises a radiation detector about every 1 cm in each plane of the phantom;
the array of radiation detectors comprises diode detectors; and
the phantom consists essentially of the solid water material.

9. The device of claim 1, wherein:
the array of radiation detectors comprises diode detectors; and
the phantom consists essentially of the solid water material.

10. The device of claim 1, wherein:
the phantom consists essentially of the solid water material; and
the array of radiation detectors comprises a radiation detector about every 1 cm in each plane of the phantom.

11. The device of claim 1, wherein:
the array of radiation detectors comprises a radiation detector about every 1 cm in each plane of the phantom;
the array of radiation detectors comprises diode detectors; and
the phantom consists essentially of the solid water material.

12. The device of claim 1, wherein the solid water material comprises 2.9-3.3% w/w glass micro bubbles, 60-90% w/w epoxy, acrylic, or polyurethane, 3-5% w/w CaCO₃, 1-3% w/w MgO, and 8-12% w/w polyethylene.

13. The device of claim 1, wherein the solid water material comprises 3.09% w/w glass micro bubbles, 57.88% w/w araldite, 23.15% w/w jeffamine, 3.89% w/w CaCO₃, 1.80% w/w MgO, 9.98% w/w polyethylene, and 0.2% w/w Na₅Al₆Si₆O₂₄S₄ or Si₄O₁₀ (OH)₂Mg₃—Co₃Ca—Al, with an elemental composition of 65.81% w/w carbon, 19.36% w/w oxygen, 8.14% w/w hydrogen, 2.21% w/w nitrogen, 1.78% w/w calcium, 1.14% w/w silicon, and 1.11% w/w magnesium.

14. The device of claim 1, wherein the width increases monotonically with the height in a direction of from a beam side surface to an opposing surface.

15. The device of claim 1, wherein the device is configured to be inserted within a head of a gantry of a linear accelerator.

16. A linear accelerator having a gantry and comprising the device of claim 1 installed in a treatment head of the gantry.

17. The linear accelerator of claim 16, further comprising software to interface the device with the linear accelerator.

18. A method for analyzing a dose response depth or a profile of a beam from a linear accelerator, the method comprising injecting the beam from a linear accelerator into the device of claim 1 and detecting the beam with the array of radiation detectors to obtain dose response depth or profile data from the beam.

19. The method of claim 18, further comprising comparing the obtained dose response depth or profile data to a treatment plan for a patient.

20. The method of claim 18, wherein:
the array of radiation detectors comprises a radiation detector about every 1 cm in each plane of the phantom;
the array of radiation detectors comprises diode detectors; and
the phantom consists essentially of the solid water material.
